(19) United States
(12) Patent Application Publication
(10) Pub. No.: US 2026/0211631 A1
(43) Pub. Date: Jul. 23, 2026

(54) CONTAINER-BASED NONLINEAR PARAMETER OPTIMIZATION SYSTEM USING R LANGUAGE AND DOCKER FOR PHARMACOKINETICS AND PHARMACODYNAMICS RESEARCH

(71) Applicant: THE INDUSTRY & ACADEMIC COOPERATION IN CHUNGNAM NATIONAL UNIVERSITY (IAC), Daejeon (KR)

(72) Inventors: Woojin JUNG, Seoul (KR); Hyunjung LEE, Daejeon (KR); Hwi-yeol YUN, Daejeon (KR); Jung-woo CHAE, Daejeon (KR)

(73) Assignee: THE INDUSTRY & ACADEMIC COOPERATION IN CHUNGNAM NATIONAL UNIVERSITY (IAC), Daejeon (KR)

(21) Appl. No.: 19/102,032

(22) PCT Filed: Jul. 11, 2023
(86) PCT No.: PCT/KR2023/009829

§ 371 Date: Feb. 7, 2025

(30) Foreign Application Priority Data
Aug. 10, 2022 (KR) 10-2022-0100088

(51) Int. Cl. G06F 8/33; G06F 8/61

(52) U.S. Cl. CPC G06F 8/33; G06F 8/63

(57) ABSTRACT

The present invention relates to a platform for providing pharmacological information of a material and, more particularly, to an R language, a docker, and a nonlinear parameter optimization system capable of providing information required for research on verifying pharmacokinetic or pharmacodynamic properties of a drug during a drug development stage. In particular, the present invention relates to a container-based integrated nonlinear parameter optimization system that gathers pieces of software required for parameter optimization research such that a pharmacometrics technology suitable for performing a mathematical task can be used and computer resources can be efficiently used for pharmacometrics research that requires high computer performance, allows the software to be immediately called under an integrated development environment to enhance a working speed of pharmacokinetics/pharmacodynamics research, enables research progress to be determined in real time through visualization and control of monitoring, and supports cooperation and remote working.

### TECHNICAL FIELD

The disclosure relates to a platform for providing pharmacological information during a drug development stage and, more specifically, to a container-based system integrating R language, a Docker, and nonlinear parameter optimization software, which is capable of providing information necessary for verifying the pharmacokinetic or pharmacodynamic properties of a drug.

In particular, the disclosure relates to a container-based integrated nonlinear parameter optimization system configured to gather pieces of software required for parameter optimization research such that a pharmacometrics technology suitable for performing a mathematical task can be used and computer resources can be efficiently used for pharmacometrics research that requires high computer performance, allow the software to be immediately called under an integrated development environment to enhance a working speed of pharmacokinetics/pharmacodynamics research, and enable research progress to be identified in real time through a visualization/control unit taking in charge of monitoring.

### BACKGROUND ART

Pharmacology is a field that studies the phenomena occurring to a drug when it is administered into the body, including metabolic pathways of drugs within the body, and reactions that occurs from the moment a drug is administered until it is completely eliminated from the body.

In pharmacology, various pieces of software are being applied in drug development. During the drug development stage, parameter optimization research to identify the pharmacokinetic/pharmacodynamic properties of a drug requires multiple pieces of software to run simultaneously, and since workflows and result processing methods are not protocolized, computer resources and labor are being used inefficiently.

Korean Patent No. 10-2146602 (registered on Aug. 13, 2020; hereinafter referred to as “Prior Art 1”) presents a method and device for providing a container-based deep learning development platform using a Docker. Prior art 1 includes the steps of receiving a service request related to deep learning from a subject, generating a Docker image in response to the service request, and generating a virtualized cloud computing environment on a deep learning server, based on the Docker image. This prior art 1 is similar in that it involves a platform using a Docker, but it lacks the necessary format for optimization tasks related to drug development as a platform for deep learning.

Korean Patent No. 10-1987664 (registered on Jun. 4, 2019; hereinafter referred to as “Prior Art 2”) presents a method for monitoring multiple clusters and applications on a cloud platform. In prior art 2, multiple container clusters and service applications operating within the clusters are comprehensively monitored, facilitating easy management. This prior art 2 is similar in that it allows container-based applications to operate on various infrastructures in a cloud platform system, but has the limitation of only observing applications as a management and supervision means.

As described above, although systems that applied a Docker to deep learning platforms have been provided, there was no system that applied the Docker to the field of drug development. Particularly, applying a Docker to drug development requires parameter optimization tasks, yet systems having a format to perform these tasks have not been provided. This results in prolonged drug development timelines.

Therefore, the need has arisen for an improved system that applies Docker technology to the drug development field while reducing the time required for drug development.

### DISCLOSURE OF INVENTION

### Technical Problem

The disclosure was developed to solve the problems in related art as described above,
and is to provide a container-based system integrating R language, a Docker, and nonlinear parameter optimization software, which facilitates various forms of collaboration and remote work during the drug development stage, thereby reducing the time and labor required for drug research.

In particular, the disclosure is to provide a system capable of reducing the time and labor required for drug research by constructing, as a web application, an information provision system for research on verifying pharmacokinetic or pharmacodynamic properties of a drug in the drug development stage by integrating software for nonlinear parameter optimization into a cloud-based container.

In addition, the disclosure is to provide a system construction method that allows for the elimination of inter-process linkage work in drug pharmacokinetic/pharmacodynamic property identification research, decentralizes a part that requires a large amount of computer calculation through web application hosting, and visualizes and controls nonlinear parameter optimization results, so as to implement a user-friendly work environment that can be utilized immediately without being limited by the type of terminal, thereby reducing the waste of secondary time required for setting up the research environment.

### Solution to Problem

In order to solve the purpose described above, the disclosure
provides a container-based nonlinear parameter optimization system using a management server including a platform container configured to provide software necessary for pharmacometrics research or pharmacokinetics/pharmacodynamics research to a plurality of connected user terminals, and the management server includes the steps of: receiving a hosting request related to a web application from a connected user terminal; generating a Docker image in response to the hosting request; implementing a working environment between the container and each terminal by using the docker image; implementing environment of the web application; executing a computer language; performing nonlinear parameter optimization; performing visualization and control; and implementing an integrated development environment.

The platform container includes a web application, and it is preferable that the web application includes an integrated development environment unit, a computer language execution unit, and a nonlinear parameter optimization unit.

The integrated development environment unit preferably includes a visualization/control unit.

The Docker image preferably configures a virtual environment within the platform container such that necessary software can be installed on Linux, which is an operation system.

The visualization/control unit preferably interacts with the parts for the computer language execution unit and parameter optimization unit by issuing commands to a terminal connected to the operation system of the integrated development environment unit.

### Advantageous Effects of Invention

The container-based nonlinear parameter optimization system utilizing R language and a Docker for pharmacokinetics/pharmacodynamics research according to the disclosure
has the effect of increasing research efficiency by increasing accessibility to minimize the time required for research, visualizing and immediately providing information required for pharmacometrics research, and enabling integrated control by linking with other software.

### BRIEF DESCRIPTION OF DRAWINGS

FIG. 1 illustrates a system configuration for implementing a method of constructing a container-based nonlinear parameter optimization system using R language and a Docker for pharmacokinetics/pharmacodynamics research according to the disclosure.

### BEST MODE FOR CARRYING OUT THE INVENTION

The disclosure may be implemented by making various modifications, and specific embodiments are illustrated in the drawings and described through detailed description. However, this is not intended to limit the disclosure to specific embodiments, but should be understood to include all modifications, equivalents, or substitutes included in the spirit and technical scope of the disclosure.

Similar reference numerals are used for similar components while describing each drawing. In describing the disclosure, detailed descriptions of related known technologies are omitted when it is determined that such descriptions may obscure the gist of the disclosure.

The disclosure may increase research efficiency by increasing accessibility to minimize the time required for research, visualizing and immediately providing information required for pharmacometrics research, and enabling integrated control by linking with other software.

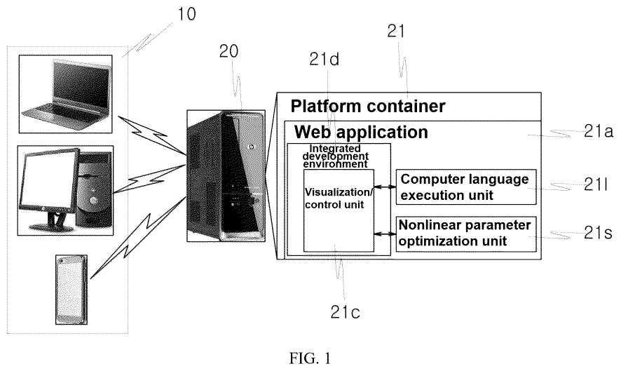

The disclosure provides a container-based nonlinear parameter optimization system utilizing R language and a Docker for pharmacokinetics/pharmacodynamics research, as illustrated in FIG. 1,
the container-based nonlinear parameter optimization system using a management server 20, including a platform container 21 configured to provide software necessary for pharmacometrics research or pharmacokinetics/pharmacodynamics research to a plurality of connected user terminals 10, the management server including the steps of: receiving a hosting request related to a web application from a connected user terminal; generating a Docker image in response to the hosting request; implementing a working environment between the container and each terminal by using the docker image; implementing environment of the web application; executing a computer language; performing nonlinear parameter optimization; performing visualization and control; and implementing an integrated development environment.

The step of receiving a hosting request refers to a step in which the management server equipped with the platform container (21) performs hosting by using a web server provided by a large telecommunications company or a specialized company.

The step of generating an image refers to a step of inputting a Docker image requested by a hosting service provider, includes information for executing the container, and includes outputting a Docker image in response to a service requested by the management server.

The step of implementing a working environment between the terminals includes receiving a service request related to deep learning from the management server and generating a virtualized cloud computing environment suitable for the requested service through a Docker.

The step of implementing web application environment refers to a step of enabling pharmacological information to be effectively implemented on the web, and includes generating a virtualized cloud computing environment, based on a Docker image, and generating and modifying the web application environment, thereby providing accurate and rapid pharmacological information, which is the purpose of the disclosure.

The step of executing a computer language refers to a step of executing a computer language, which is a language that instructs a computer on a method and sequence of processing tasks to solve a problem, and may provide various development languages and development environments.

The step of performing nonlinear parameter optimization includes optimizing pharmacokinetics/pharmacodynamic problems expressed as nonlinear functions, by utilizing R language and parameter optimization software. In other words, the step of performing nonlinear parameter optimization includes gathering nonlinear parameter optimization programs necessary for performing a mathematical task for pharmacological research and providing the programs within a web application, thereby immediately calling the programs and enhancing the work speed.

The step of performing visualization and control enables the progress of the research to be identified in real time through visualization and control responsible for monitoring.

The step of implementing an integrated development environment includes configuring an environment enabling smooth access for users connected via a web browser, regardless of the system type or operation system that constitutes the management server.

The platform container 21 constitutes a system for providing pharmacological information via the web, as described above, and includes a web application 21a to perform each of the steps, and the web application 21a includes an integrated development environment unit 21e, a computer language execution unit 211, and a nonlinear parameter optimization unit 21s.

The integrated development environment unit 21e includes a visualization/control unit 21c.

As described above, the Docker image configures a virtual environment within the platform container to allow pieces of necessary software to be installed on Linux, which is an operation system.

The visualization/control unit may issue a command to the terminal connected to the operation system of the integrated development environment unit. In the system construction configured as described above, a researcher may register a Docker image on his/her terminal 10 to host a server, access the web application unit 21a, and access the inside of the corresponding container 21. The visualization/control unit 21c may be mounted to an upper layer of integrated development environment unit 21e and may operate in parallel with the integrated development environment unit to communicate with the computer language execution unit 211 and the nonlinear parameter optimization unit 21s.

The visualization/control unit may control the operation of the computer language unit and the nonlinear parameter optimization unit by performing the task in the integrated development environment provided by the integrated development environment unit, and may monitor the computer resources and progress allocated to the optimization operation.

Finally, after providing the visualized results to the researcher, the researcher repeats the process described above to perform the optimization research.

The user terminal 10 is not limited to a computer, and may be any device that is able to access a web page.

The platform container 21 may refer to a virtual operation system on which an application is executable, and the web application provided by the web application unit may provide R language and development environment and include htmltools, reactable, rstudioapi, shiny, xpose, ggplot2, etc.

The integrated development environment unit 21d includes R studio, which is representatively used in the R language, the visualization/control unit 21c may include the Shiny program, and the computer language execution unit 211 may include Perl speaks NONMEM (PsN), R, etc.

The nonlinear parameter optimization unit 21s may include NONMEM, etc.

### DESCRIPTION OF REFERENCE NUMBERS

10: User terminal
20: Management server
21: Platform container
21a: Web application unit
21d: Integrated development environment unit
21s: Nonlinear parameter optimization unit
21c: Visualization/control unit
211: Computer language execution unit

1. A container-based nonlinear parameter optimization system utilizing R language and a Docker for pharmacokinetics/pharmacodynamics research, the container-based nonlinear parameter optimization system using a management server including a platform container configured to provide software necessary for pharmacometrics research or pharmacokinetics/pharmacodynamics research to a plurality of connected user terminals, wherein the management server comprises the steps of:
receiving a hosting request related to a web application from a connected user terminal;
generating a Docker image in response to the hosting request;
implementing a working environment between the container and each terminal by using the Docker image;
implementing environment of the web application;
executing a computer language;
performing nonlinear parameter optimization;
performing visualization and control; and
implementing an integrated development environment.

2. The container-based nonlinear parameter optimization system of claim 1,
wherein the platform container comprises a web application, and
wherein the web application comprises an integrated development environment unit, a computer language execution unit, and a nonlinear parameter optimization unit.

3. The container-based nonlinear parameter optimization system of claim 2,
wherein a visualization/control unit is further included in the integrated development environment unit, and
wherein the visualization/control unit is connected in parallel to the computer language execution unit and the nonlinear parameter optimization unit.

4. The container-based nonlinear parameter optimization system of claim 1, wherein the Docker image configures a virtual environment within the platform container so as to allow pieces of necessary software to be installed on Linux which is an operation system.

5. The container-based nonlinear parameter optimization system of claim 3, wherein the visualization/control unit is configured to interact with parts for the computer language execution unit and parameter optimization unit by issuing a command to a terminal connected to the operation system of the integrated development environment unit.
